(19) United States
(12) Patent Application Publication
(10) Pub. No.: US 2017/0065469 A1
(43) Pub. Date: Mar. 9, 2017

(54) FIRST AID INSIDE ASSEMBLY CARRIER SYSTEM

(71) Applicant: Richard F. E. Newcombe, Plano, TX (US)

(72) Inventor: Richard F. E. Newcombe, Plano, TX (US)

(21) Appl. No.: 14/847,073

(22) Filed: Sep. 8, 2015

(51) Int. Cl. A61F 17/00

(52) U.S. Cl. CPC A61F 17/00

(57) ABSTRACT

A system for organizing and attaching essential first aid items to a carrier suitable for insertion to and extraction from various housings or containers typically included as essential items for personal care or equipment used in sporting, camping, hiking, travel and other recreational endeavors; which, includes a readily identifiable marking label to include notification of contents within such housings or containers and is specifically intended for insertion into items or equipment for a participant to actively carry or use in the particular activity.

### CROSS-REFERENCE TO RELATED APPLICATION 35 USC §119(e)

Not Applicable

### FIELD OF INVENTION

This invention relates to organizers and the assembly of discrete first aid and survival components connected to a carrier system which is conveniently inserted into other objects such as a tube, housing or container. The ease by which the components are extracted from the housing or container is particularly of importance as the system anticipates primary use during times of emergency when accessibility is impaired due to injury of the user and is being operated under stressful conditions which sometimes will constitute a personal injury emergency. Furthermore, the carrier system is required to provide secure attachment of discrete components to the carrier system to prevent inadvertent loss of a component when deployed and which will ultimately insure components are available and within reach when needed. Additionally, the carrier system is organized during assembly to configure the first aid components in a logical sequence of administration by the user providing a sequential presentation of first aid components when retrieving the carrier from the container, or while opening the packaging presented, i.e. antiseptic wipes first, ointment next and bandages last.

### BACKGROUND OF INVENTION

Safety and Survival markets are enjoying unprecedented popularity and media exposure. The principal activity First Aid Inside Assembly Carrier System customers will engage in are walking and hiking and other outdoor activities. Though they are a small subset of outdoor activities, that group has grown to over 44 million people in America last year; and, the number of regular participants continues to climb annually.

The number of people engaging in outdoor activities is increasing along with the frequency of those activities. And while hiking becomes less popular by age, walking becomes more popular as baby boomers are aging. Additionally, an aging American population is living longer. As such, simple exercise (walking) aided by walking sticks or canes will continue to rise in popularity as Baby Boomers resist the “immobility” of aging.

First Aid Inside Assembly Carrier System products belong to two categories that are both enjoying long term positive growth. The first category is walking sticks and the second is first aid pouches. First Aid Inside Assembly Carrier System will enter the market with a design and deployment configuration suitable for both.

In the first example, the First Aid Inside Assembly Carrier System is configured for insertion into the hollow tube structure of the typical walking cane. The First Aid Inside Assembly Carrier System can be inserted into this void to allow hikers and walkers to carry essential first aid components while hiking or walking. Having the First Aid Inside Assembly Carrier System contained within the walking stick will ensure first aid components are readily available when needed. Also, the user will be less likely to forget to pack a first aid kit with them when they embark on their route due to this convenient method as presented herein. The present invention envisions manufacturing walking sticks containing the First Aid Inside Assembly Carrier System for point of sale to consumers.

In the second example, the First Aid Inside Assembly Carrier System is customizable for insertion into any container which the consumer desires to carry first aid components. The use of flexible materials and variable length assembly features of the First Aid Inside Assembly Carrier System will allow consumers to insert the First Aid Inside Assembly Carrier System into shoulder straps, belts, pockets, bags, pouches or other similar gear which a person might carry with them while performing an activity.

Safety and survival favors the prepared and that's why First Aid Inside Assembly Carrier System is here. And yet it seems that every year we read about people, families and even Boy Scouts getting lost outdoors for days—and, while most stories end well, some do not. The First Aid Inside Assembly Carrier System provides essential first aid components which are likely to assist a person in need of first aid via availability and accessibility of first aid components conveniently when needed.

For more than 100 years, inventors have developed various concepts for carrying first aid components upon a person. In 1911, inventors sought to provide a receptacle which would attach to a belt for carrying purposes. As recently as 2012, inventors introduced a new way to organize and carry first aid components. The present invention acknowledges all of these many different and novel approaches to carrying first aid components and improves upon them by introducing a First Aid Inside Assembly Carrier System which provides a novel carrier system to organize and contain the first aid components in addition to a unique outside labeling device to alert people as to the contents within a multitude of container devices.

In 1911, U.S. Pat. No. 1,007,515 which offered a Carrier for First-Aid Packages or Outfits was presented wherein the object of the invention was to provide a receptacle for this purpose for attachment to a cartridge-belt. Here, the inventor focused the teaching on a pockets attached to a belt in which first-aid packages would be carried. The present invention differs from the pockets described by providing a carrier system that provides for attachment of the individual first aid components to a carrier system which when assemble could fit into such a pocket.

In 1916, U.S. Pat. No. 1,210,377 introduced weaving patterns and techniques for constructing the pockets and to provide for reduced thickness of the pocket, greater capacity to each pocket, and flexibility in attaching the pocket to a person's outer wear. While the present invention addresses the attachment of first aid components to a carrier system for insertion into a container, the illustration present in U.S. Pat. No. 1,210,377 suggests that containment of such individual first aid component has always been important and that flexibility in containment is desirable.

In 1920, U.S. Pat. No. 1,339,795 introduced pocket cover flaps to provide improved retention and accessibility features of the carrier system deployed. The present invention differs again by providing a mechanism by which the user can easily extract the contents of a carrier such as this and avoid inadvertent dropping or loss of the discrete first aid components when the carrier is opened and the user begins to retrieve a first aid component.

Nearly 100 years later, US 2002/0104560 A1 introduce a Fish Stick and Survival Stick constructed using five pieces of aluminum and a cloth inside liner. Essentially, the Survival Stick presented therein illustrates both the need to carry first aid components and the desire to do so in a multi-function device such as a walking stick. While such a Survival Stick exists, the problem of individual first aid components getting lost or dropped when the user extracts the cloth liner remains at issue, the solution for such a problem is addressed by the present invention. Additionally, absent an external marking such as provided with the present invention, the utility of having first aid components readily available might be over looked by a third party assisting a person in need who is unaware of the contents within the stick.

In 2006, EP 1 769 782 A2, provided a capsule to contain various first aid components which then could be placed inside a container such as a pocket, pouch or in this specific example, a special stick. In this example, the capsule is specifically designed in shape and size to carry a limited number of first aid components and only those components envisioned by the shape and size provided. The present invention differs by providing greater flexibility to the total quantity of discrete first aid components to be attached to the carrier system, the variable length of the carrier system provided by the present invention by way of attaching multiple carrier systems together, and the individualization of the first aid components to be attached and carried based on the risks and potential exposures the user may encounter in a particular activity.

In 2012, CN 202959092 U and CN 202980452, continued development of cavity based stick containers continue to emerge and improve upon these prior inventions. But like in the previous discussion, the present invention addresses the need to attach the discrete first aid components to a carrier system which can be inserted into such a cavity for the reasons outlined above.

Therefore, although there are several apparatuses which may have various functions related to the First Aid Inside Assembly Carrier System, none of these either separately or in combination with each other, teach or anticipate the current invention. Therefore, there remains an unmet need in the field of inserting, containing and providing controlled access to and retention of discrete first aid components along with familiar external labeling to which is convenient to users in somewhat isolated circumstances where personal injury may occur. The current invention will fulfill this unmet need.

### SUMMARY OF INVENTION

The following presents a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

When the user of the First Aid Inside Assembly Carrier System undertakes preparation for a activity, a risk assessment is performed regarding location, season, weather exposure and personal needs. Some activities require formal risk assessments and mitigating preparations to ensure the well-being and overall safety of the person. Mountain climbing, desert riding, off-shore boating are just a few examples. Other activities such a walking in the neighborhood, biking along city trails and spectator sports require less formal risk assessments and preparations. While still other activities such as hiking in the forest, exploring caves and overnight camping or hunting private land require some level of risk assessment which includes preparation for emergencies which includes intentionally carrying first aid.

The invention described herein provides a convenient system to incorporate carrying first aid conveniently within the equipment likely to be “in front of mind” when embarking on various activities. The basic premise is to provide a carrier system which the user can insert into the ordinary attire one would wear or the equipment one would use to perform the activity. As such, anytime the user undertakes to engage in the activity by getting dressed or gathering basic equipment, the First Aid Inside Assembly Carrier System is there with them.

First Aid Inside Assembly Carrier System is an apparatus that is used to insert and retrieve first aid components from within the apparel or equipment used in a particular activity. The essential features of the First Aid Inside Assembly Carrier System are attachments which connect the discrete first aid component to the carrier system; the flexibility to insert or retrieve the carrier system from a container by using grasping tabs on the carrier system; the retention of discrete first aid components to the carrier system when removed from the container to prevent inadvertent loss resulting from spilling or dropping loose filled items into a container; and, the users discretion to select which first aid components to attach to the carrier system, and in which order to sequence the attachments to the carrier system for the exposures anticipated during the particular activity being pursued.

There are several methods currently used today to carry first aid kits. The present invention does not alter or disable this functionality. Essentially, the First Aid Inside Assembly Carrier System adds functionality to any existing first aid kit or carrying device. This is accomplished by introducing the features outlined above.

The First Aid Inside Assembly Carrier System specifically anticipates other inherent limitations found in the existing art. First, the current methods for carrying first aid components are potentially inaccessible during an emergency. For example, if a person is injured and has limited use of arms, hands or fingers preventing reach to a pocket or container to extract the first aid components desired, the first aid sought is unavailable. Also, if a third party comes upon an injured person and is unaware of the first aid components placed inside a pouch, walking stick or other container, the first aid sought is unavailable. The First Aid Inside Assembly Carrier System shown in the present invention address both issues as described below.

To resolve the problems mentioned above, an object of the present invention is to provide a flexible carrier system which can be inserted during production or post production, without disabling the inherent safety functions or functionality of the apparel or equipment selected by the user to contain the first aid components.

It is another object of the present invention to provide the user a particular benefit of “awareness” which is found in the present invention. This benefit is realized when the user applies a corresponding sticker label First Aid Inside™ to the container which is readily recognizable as to First Aid Inside™.

It is yet another object of the present invention to provide the user options as to the first aid components to be carried. The present invention provides for attachment points to securely attach a discrete first aid component to the carrier system in a sequence that is determined by the user. This feature allows the user to customize the first aid inventory contents within the container. Such inventory may be limited to medication components, or may include essential nourishments such as vitamins, minerals, water purification tablets, protein, and other similar items.

It is yet another object of the present invention to provide for modification of the length of the First Aid Inside Assembly Carrier System by providing connection points on each segment of the carrier system. This feature of the present invention provides users greater flexibility in distributing contents across various apparel or containers in their possession. As such, the user can duplicate essential components in multiple locations within their clothing and within the equipment they use. Alternatively, segments of the carrier system can be attached together to form a longer carrier system to be inserted into a larger container.

The present invention comprises a carrier spine with attachment points which can be constructed of rigid or flexible material. A rigid material can be used inside a hollow tube container; while a flexible material, even a segment of string, can be used inside hollow tubes or when necessary to coil the carrier system to fit within a smaller container or pouch.

The attachment points provide a mechanism to attach discrete first aid components to the carrier spine. These attachment points can be hook and loop, snap, magnetic, adhesive or tape techniques to secure each discrete component to the carrier spine, arranged in sequence along the spine at the discretion of the user. The simplest mode of attachment is glue. Such arrangement could follow a prescribed sequence of application of first aid to assist the user in applying first aid appropriately, presenting each component in sequence so as also allow the user to self-administer the first aid while impaired by circumstances or by injury.

Finally, the present invention provides enhanced awareness of available first aid components contained within an article of clothing or within the equipment present. This awareness is presented as a familiar adhesive label designed to be visible and recognizable as to the location of First Aid Inside™.

Still other objects of the present invention will become readily apparent to those skilled in this art from the following description wherein there is shown and described the embodiments of this invention, simply by way of illustration of the best modes suited to carry out the invention. As it will be realized, the invention is capable of other different embodiments and its several details are capable of modifications in various obvious aspects all without departing from the scope of the invention. Accordingly, the drawing and descriptions will be regarded as illustrative in nature and not as restrictive.

### BRIEF DESCRIPTION OF THE DRAWINGS

Various exemplary embodiments of this invention will be described in detail, wherein like reference numerals refer to identical or similar components, with reference to the following figures, wherein:

FIG. 1 is a perspective view of the First Aid Inside Assembly Carrier System horizontally positioned showing the first end connection point, followed by a sequential distribution of first aid components positioned in the likely order of use, each spaced and individually attached to the Carrier System.

FIG. 2 is a perspective view of preferred embodiment wherein each individual first aid component is affixed to the Carrier System using string and glue as the method for securing the first aid component to the Carrier System.

FIG. 3 is a perspective view of preferred embodiment structure wherein a magnet is attached to the first end of the Carrier System.

FIG. 4 is a perspective view demonstrating the use of a magnet as a device which enables the user to attach the Carrier System to the users clothing when deploying the First Aid Inside Assembly Carrier System.

FIG. 5 is a perspective of the preferred embodiment of the First Aid Inside Assembly Carrier System for insertion into a walking stick.

FIG. 6 is a perspective of the preferred embodiment of the “First Aid Insider™ ” label.

FIG. 7 is a perspective view of the preferred embodiment of the First Aid Inside Assembly Carrier System coiled for insertion into a pouch.

### DETAILED DESCRIPTION

The claimed subject matter is now described with reference to the drawings. In the following description, for purposes of explanation, numerous specific details are set forth in order to provide a thorough understanding of the claimed subject matter. It may be evident; however, that the claimed subject matter may be practiced with or without any combination of these specific details, without departing from the spirit and scope of this invention and the claims.

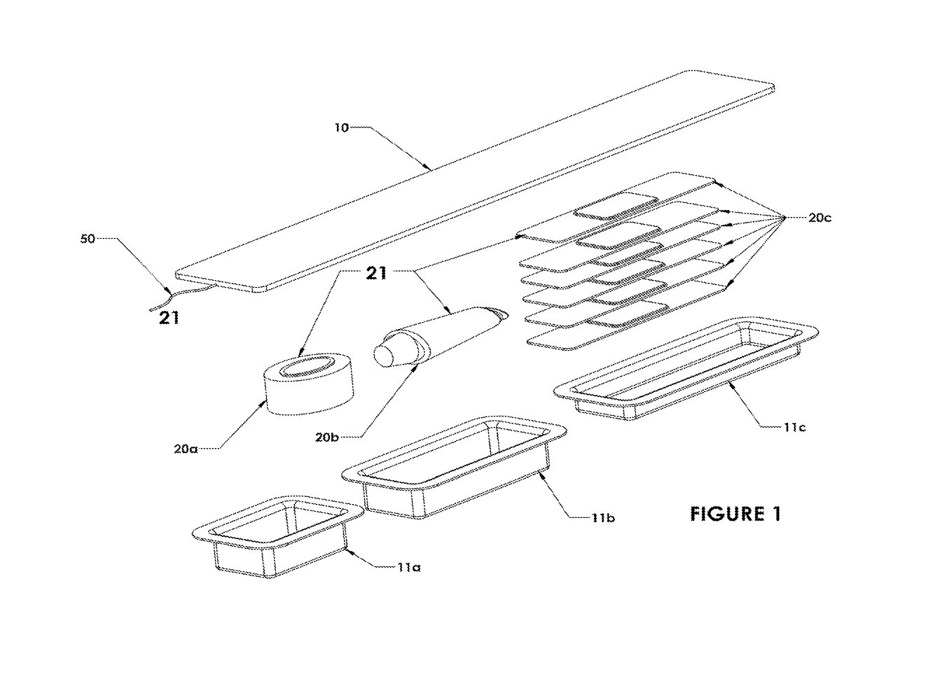

FIG. 6 illustrates the First Aid Inside Assembly Carrier System 10 assembled for insertion into an elongated carry device such as a tube, walking stick or other similar form. The First Aid Inside Assembly Carrier System 10 in FIG. 1 enables the user to organize first aid components 20 in a illustrative non-limiting logical sequence 21 of application during treatment for injury.

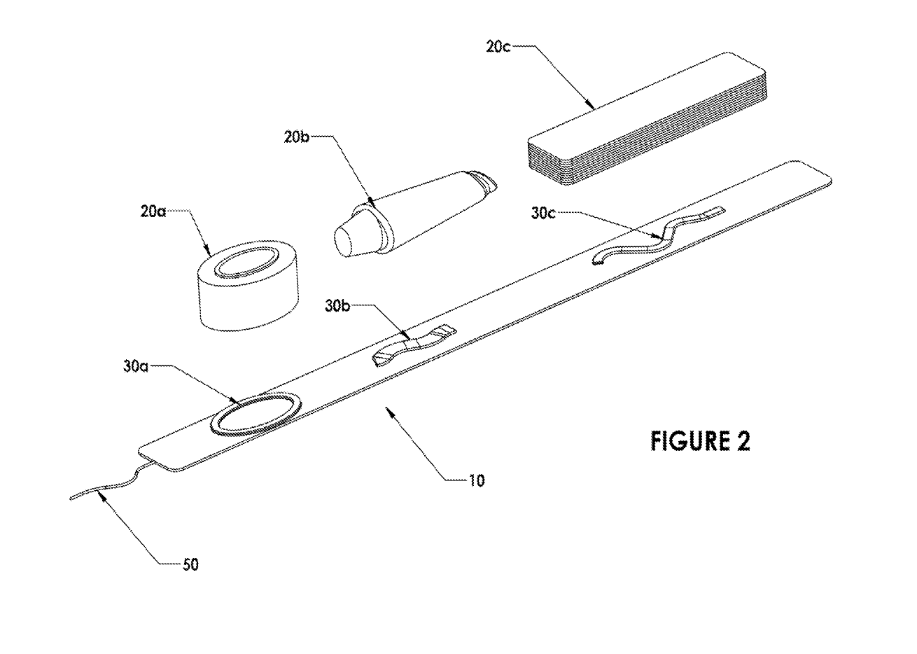

The First Aid Inside Assembly Carrier System 10 provides users a systematic approach to carrying, retrieving and applying first aid. First, the carrier system 10 provides the user a system to organize the first aid components 20 according to a sequence 21 and various attachment points 30 as a system to retain the components 20 during retrieval and application. In the past, first aid components 20 have been inserted into pouches or tubes, yet when the pouch or tube is opened, the components 20 were loosely gathered and unorganized. As such, the user could easily drop and contaminate the component 20 when attempting to unpack and apply first aid. The current embodiment overcomes this risk by securely attaching each first aid component 20 to the carrier system 10 in a preferred sequence 21 in order of application preference during the administration of first aid using various attachment points 30 as shown in FIG. 2.

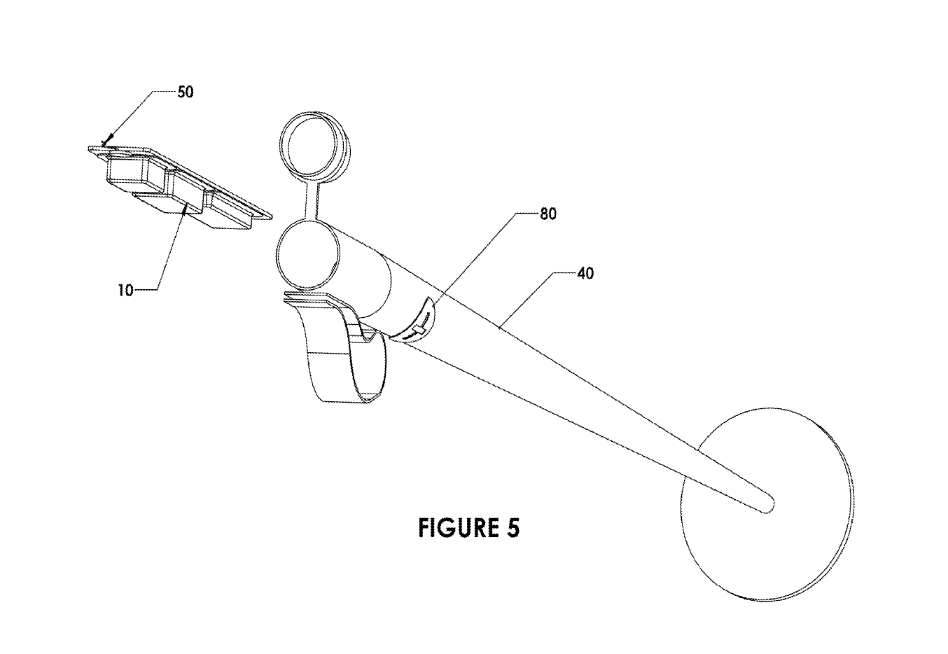

The carrier system 10 itself provides a means to insert and extract the first aid components 20 from the container 40 in FIG. 5 in which the First Aid Inside Assembly carrier system 10 is placed. By having the first aid components 20 attached to a carrier system 10, the assembled sequence 21 of components 20 has form and structure to slide into a tube 40, hollow stick or pouch 70 FIG. 7 while retaining the organizational integrity of the assembly. A device such as a string 50 FIG. 5 can be attached to either or both ends of the carrier system 10 to assist the user in inserting or extracting the assembly when necessary. This feature of the carrier system 10 simplifies inserting multiple first aid components 20 into a carry device 40. This feature also simplifies the extraction process while also ensuring first aid components 20 do not get stuck inside the carry device 40 when extracting the carrier system 10 with attached components 20.

FIG. 2 illustrates the preferred present embodiment comprised of the first aid components 20 glued, where glue is one method for attachment, at attachment point 30 to the Carrier System 10. When the user extracts the carrier system 10 from the container 40 into which the carrier system 10 was inserted, the organized assembly is presented to the user with the first logical component. The user will then grasp and pull the component 20 from the carrier system 10 or open the optional packet cover 11 in FIG. 1 by pulling the packet tab 26 to retrieve the first aid component 20 for application to a wound. Next, the user will retrieve and apply the next component 20 and repeat as necessary.

The optional packet cover 11 in FIG. 1 provides additional functionality to the Carrier System 10 to protect the components 20 from natural elements and environmental conditions such as rain, humidity, sand, and other contaminants.

The carrier system 10 ensures the first aid components 20 are retrieved from the carrying container 40, that all components 20 are retained, and that components 20 are accessible within reach of the user holding the carrier system 10.

The sequence 21 in which first aid components 20 are assembled on the carrier system 10 is at the discretion of the user. While the First Aid Inside Assembly Carrier System 10 will come pre-assembled based on recommendations for application of first aid, the user has the ability to reorganize the sequence 21 to their individual preference.

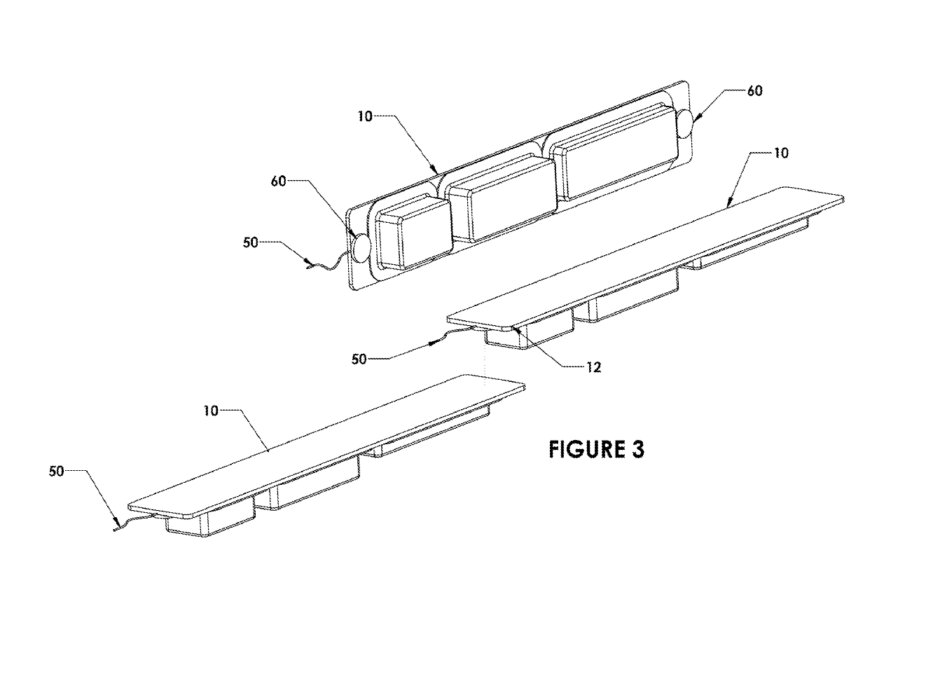

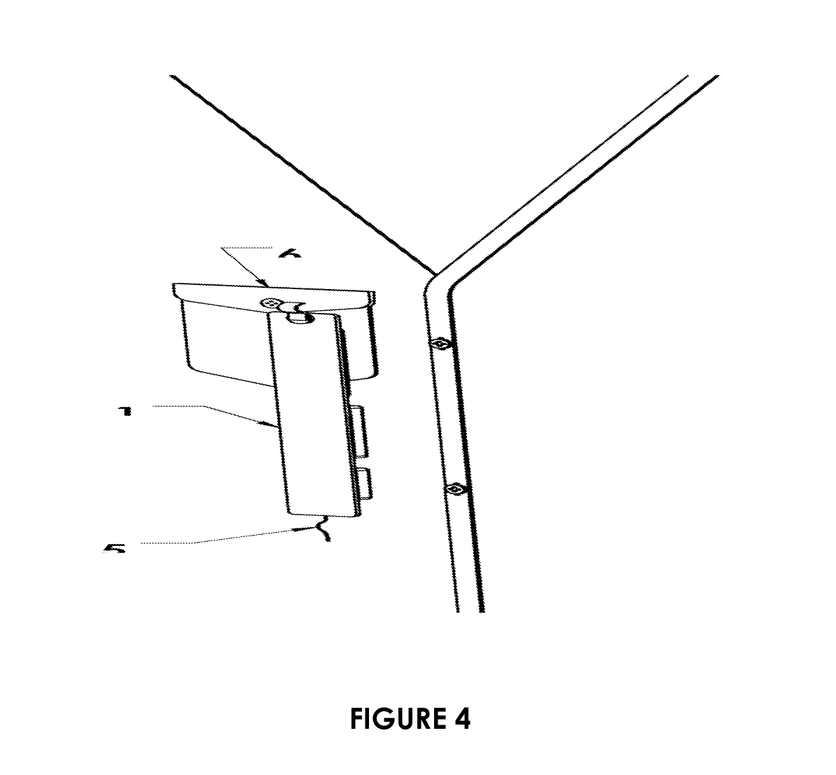

In FIG. 3, the First Aid Inside Assembly Carrier System 10 is shown with a magnet 60 affixed to the leading end 12 of the Carrier System 10. The magnet 60 could be substituted with a button hole, snap, loop and hook material or any other preferred method for attaching the Carrier System 10 to the users clothing 65 FIG. 4 or body.

The function of the magnet 60 or alternative attachment method is to provide the user with an attachment device to secure the carrier system 10 to the user when the carrier system 10 is extracted from the container 40. The purpose of this attachment is to prevent or lessen the risk of dropping the carrier system 10 when applying first aid. When the carrier system 10 is extracted from the container 40 the user would attach the assembly to one's person or clothing 65 FIG. 4. This attachment secures the carrier system 10 for retention and provides the user leverage in detaching first aid components 20 from the carrier system 10 during the application of first aid. Since is it likely the user will require application of first aid to oneself, this feature aids the user in applying first aid by enabling the user to use only one hand to retrieve and apply first aid components 20 from the carrier system 10.

In FIG. 7, the First Aid Inside Assembly Carrier System 10 is shown coiled for insertion into a pouch, pocket, bag or other container 70 other than a tube or hollow walking stick 40. The features of sequencing, retention, and attachment are preserved when coiled as the structural support of the carrier system 10 is flexible.

The First Aid Inside Assembly Carrier System 10 is to be deployed in conjunction with an external label “First Aid Inside™” 80 shown in FIG. 6. Deploying the First Aid Inside Assembly Carrier System 10 in conjunction with the label 80 accomplishes several objectives. First, the user provides greater awareness to others who might render first aid to the user that first aid components 20 are contained with the equipment and tools carried by the user at the time. Such awareness and visibility can quicken the access to and application of first aid to a person.

Also, the label 80 and Carrier System 10 can be applied to and inserted into carrying devices most likely to be used by the user during the activity. If the user elects to insert the carrier system 10 and apply the label 80 to any essential equipment used in performing the activity, such as a walking cane 40 used during hiking, the user is less likely to forget to carry the equipment containing the carrier system 10 with them.

The preferred embodiment encompasses the First Aid Inside Assembly Carrier System 10 and First Aid Inside™ label 80 in conjunction.

The present embodiment is not restricted in use or application to only containers. The First Aid Inside Assembly Carrier System 10 is assembled and has the structural integrity to be carried by the user without a container. Accordingly, the First Aid Inside Assembly Carrier System 10 can hang on a wall, behind a door, attach to the side of a tent or be placed along the path of an activity for convenience to multiple users.

The present embodiment is not restricted in use or application to personal first aid applied to the body of a human being. The Carrier System presented herein may also be used for medications, decontamination, survival, nutrition or other exposures similar to what is described herein.

It may be advantageous to set forth definitions of certain words and phrases used in this patent document. The term “couple” and its derivatives refer to any direct or indirect communication between two or more elements, whether or not those elements are in physical contact with one another. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

What has been described above includes examples of the claimed subject matter. It is, of course, not possible to describe every conceivable combination of components or methodologies for purposes of describing the claimed subject matter, but one of ordinary skill in the art can recognize that many further combinations and permutations of such matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

We claim:

1. A first aid component carrier system which is comprised of
a. A series of discrete first aid components;
sequenced in accordance with preferred application recommendations;
connected to a carrier device;
equipped with connection points on each component and the carrier device;
which can be inserted into a housing or container;
which can be deployed by the user;
by extracting the carrier device from the housing or container by pulling on the carrier device;
to retrieve the first aid component desired;
without inadvertently dropping or losing other first aid components.

2. The carrier device claim 1, wherein the overall length of the carrier device is determined by interconnecting multiple carrier devices end to end or side by side.

3. The carrier device in claim 1, wherein the connection points are made between the carrier device at various attachments points and the discrete first aid component which provides for secure attachment of the first aid component to the carrier device, yet allows the user to access and detach the first aid component from the carrier device with relative ease.

4. The first aid component carrier system in claim 1 above wherein an optional component packaging cover is applied to separately contain each first aid component attached to the carrier system.

5. The first aid component carrier system in claim 1 above wherein an optional component packaging cover is applied to collectively contain multiple first aid components attached to the carrier system.

6. The first aid component carrier system in claim 1 above is a single use application.

7. The first aid component carrier system in claim 1 above is a reusable application.

8. The carrier device in claim 1 above, wherein a pull-cord is attached to the end of the carrier system to provide the user a grasping point to hold and pull to remove the carrier device from the housing or container.

9. The first aid component carrier system which is identified by an externally applied label marking the content of the housing or container as being equipped with essential first aid components.
